(19) United States
(12) Patent Application Publication
(10) Pub. No.: US 2023/0263777 A1
(43) Pub. Date: Aug. 24, 2023

(54) STABLE SOLID FORMULATION OF AZILSARTAN OR PHARMACEUTICALLY ACCEPTABLE SALTS THEREOF

(71) Applicant: PIRAMAL PHARMA LIMITED, Mumbai (IN)

(72) Inventors: Umesh BARABDE, Ahmedabad, Gujarat (IN); Vishakh KHARAT, Ahmedabad, Gujarat (IN); Amit JAIN, Ahmedabad, Gujarat (IN); Dhananjay SINGARE, Ahmedabad, Gujarat (IN); Mahek NAIK, Ahmedabad, Gujarat (IN); Nawanit SHARMA, Ahmedabad, Gujarat (IN)

(73) Assignee: PIRAMAL PHARMA LIMITED, Mumbai (IN)

(21) Appl. No.: 18/014,197

(22) PCT Filed: Jul. 2, 2021
(86) PCT No.: PCT/IB2021/055957

§ 371 Date: Jan. 3, 2023

(30) Foreign Application Priority Data
Jul. 3, 2020 (IN) 202021028444

(51) Int. Cl. A61K 31/4245; A61K 31/4035; A61K 9/20; A61K 9/28

(52) U.S. Cl. CPC A61K 31/4245; A61K 31/4035; A61K 9/2095; A61K 9/2013; A61K 9/2018; A61K 9/2027; A61K 9/2009; A61K 9/2054; A61K 9/28

(57) ABSTRACT

The present invention relates to a stable, solid composition of Azilsartan or its pharmaceutically acceptable salts thereof and one or more pharmaceutically acceptable excipient(s); optionally, the said composition further comprises a diuretic, preferably a thiazide diuretic such as Chlorthalidone, and processes for its preparation.

### FIELD OF THE INVENTION

The present invention provides a stable solid pharmaceutical composition comprising Azilsartan or pharmaceutically acceptable salts thereof and one or more pharmaceutically acceptable excipient(s); optionally, the said composition further comprises a diuretic.

### BACKGROUND OF THE INVENTION

Azilsartan is an angiotensin II receptor antagonist, which blocks the vasoconstriction activity of angiotensin II by selectively blocking the binding of angiotensin II and vascular smooth muscle AT1 receptor. Angiotensin II receptor antagonists are used in the management of hypertension, to lower the blood pressure. The potassium compound of Azilsartan medoxomil has the chemical name (5-methyl-2-oxo-2H-1,3-dioxol-4-yl)methyl 2-ethoxy-1-({4-[2-(5-oxo-4,5-dihydro-1,2,4-oxadiazol-3-yl)phenyl]phenyl}methyl)-1H-1,3-benzodiazole-7-carboxylate and its structural formula is as follows.

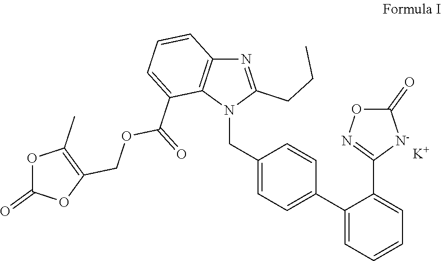 [SMILES: CCCc1nc2cccc(C(=O)OCc3oc(=O)oc3C)c2n1Cc1ccc(-c2ccccc2-c2noc(=O)[n-]2)cc1 ; [K+]]

Azilsartan kamedoxomil is a white to nearly white powder with a molecular weight of 606.62. It is practically insoluble in water and freely soluble in methanol.

Diuretics are orally administered in the treatment of hypertension and edema. Well known diuretics are thiazides which are known moderately potent diuretics and exert their diuretic effect by reducing the reabsorbtion of electrolytes from the renal tubules, thereby increasing the excretion of sodium and chloride ions, and consequently of water.

Chlorthalidone, also known as Chlortalidone, is a diuretic medication used to treat high blood pressure, swelling including that due to heart failure, liver failure, and nephrotic syndrome, diabetes insipidus, and renal tubular acidosis and its structural formula is as follows.

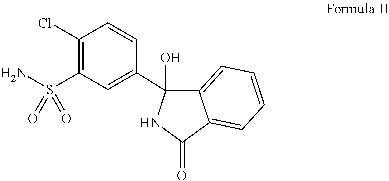 [SMILES: NS(=O)(=O)c1cc(C2(O)NC(=O)c3ccccc32)ccc1Cl]

In high blood pressure use of Chlorthalidone is a preferred initial treatment. It is also used to prevent calcium-based kidney stones. It is taken by mouth. Effects generally begin within three hours and last for up to three days.

It is known that co-administration of an angiotensin II receptor antagonist and a diuretic is an effective therapy for the prevention or treatment of hypertension.

Azilsartan kamedoxomil is marketed as tablets for oral administration under the brand name EDARBI® in strengths of 40 and 80 mg equivalent to Azilsartan medoxomil.

Azilsartan kamedoxomil in combination with Chlorthalidone is marketed as tablets for oral administration under the brand name EDARBYCLOR® in strengths of Eq 40 mg Medoxomil; 12.5 mg and EQ 40 mg Medoxomil; 25 mg.

U.S. Pat. No. 7,157,584 B1 describes Azilsartan compound specifically and it's salt.

U.S. Pat. No. 9,066,936 B1 describes composition comprising Azilsartan, a pH controlling agent which provides a pH of 3 to 5 when dissolved or suspended in water at a cons. 1% w/v at 25 degree C.

U.S. Pat. No. 9,169,238 B1 relates to a solid preparation comprising a first part comprising Azilsartan and pH controlling agent and a second part comprising Chlorthalidone which is obtained by separate granulation; pH is 2 to 5.

However, there is need to develop stable pharmaceutical composition comprising a specific angiotensin II receptor antagonist, such as compound of formula I with specific pH optionally in combination with diuretic selected from thiazide derivatives such as compound of formula II. The main challenge is to provide stable pharmaceutical composition comprising compound of formula I either alone or in combination with diuretic when the pH range is more than 5 which has an adequate content uniformity causing a good dispersion upon oral administration and high bioavailability with improved manufacturing processes and stability and a robust final dosage form for their preparation and use thereof.

Considering pH range greater than 5, composition of various drugs have been investigated and it has been found that a pharmaceutical composition containing a specific angiotensin II receptor antagonist, such as compound of formula I, and optionally one or more diuretics selected from thiazide derivatives such as compound of formula II exerts excellent anti-hypertensive effects and hence is useful as a preventative and/or therapeutic agent for hypertension.

### SUMMARY OF THE INVENTION

The present invention relates to a stable solid pharmaceutical composition comprising Azilsartan or its salt and one or more pharmaceutically acceptable excipient(s); optionally the composition further comprises one or more diuretics. It further relates to a process for preparation of the said composition, wherein the said composition is used in the treatment of hypertension by orally administering to a subject in need thereof.

In one general aspect, the present invention provides a stable solid pharmaceutical composition comprising Azilsartan or its salt and one or more pharmaceutically acceptable excipient(s); wherein the composition is used in the treatment of hypertension by orally administering to a subject in need thereof.

In another general aspect, the present invention provides a stable solid pharmaceutical composition comprising Azilsartan or its salt and one or more pharmaceutically acceptable excipient(s); wherein one or more pharmaceutically acceptable excipient(s) include but are not limited to fillers, diluents, binders, disintegrants, coating agents, glidants, surfactants, pH modifiers, lubricants, vehicle, polymer or coating system and the like.

In another general aspect, the present invention provides use of the potassium compound of Azilsartan medoxomil having compound of formula I and chemical name (5-methyl-2-oxo-2H-1,3-dioxol-4-yl)methyl 2-ethoxy-1-({4-[2-(5-oxo-4,5-dihydro-1,2,4-oxadiazol-3-yl)phenyl]phenyl}methyl)-1H-1,3-benzodiazole-7-carboxylate, in the treatment of hypertension in a subject in need thereof.

In another general aspect, the present invention provides a process for preparing a solid pharmaceutical composition comprising Azilsartan or its salt and one or more pharmaceutically acceptable excipient(s); wherein the composition is used in the treatment of hypertension by orally administering to a subject in need thereof.

In another general aspect, the present invention provides stable solid pharmaceutical composition comprising Azilsartan kamedoxomil and one or more pharmaceutically acceptable excipient(s), wherein 1% w/v solution of acid and base combination results in pH above 5, specifically in the range from 5.1 to 7.0.

In another general aspect, the present invention provides stable solid pharmaceutical composition comprising Azilsartan kamedoxomil and one or more pharmaceutically acceptable excipient(s), wherein 1% w/v solution of acid and base combination used in the present composition, gives pH range from about 5.1 to about 7.0 when dissolved or suspended in water; wherein the said composition is used in the treatment of hypertension by orally administering to a subject in need thereof.

In another general aspect, the present invention provides stable solid pharmaceutical composition comprising Azilsartan or its pharmaceutically acceptable salt thereof; wherein the said composition further comprises a diuretic; specifically the diuretic includes thiazide diuretics; more specifically the thiazide diuretic is Chlorthalidone.

In another general aspect, the present invention provides stable solid composition comprising combination of Azilsartan kamedoxomil and Chlorthalidone and one or more pharmaceutically acceptable excipient(s), wherein the composition is administered orally for the treatment of lowering the blood pressure in a subject in need thereof.

In another general aspect, the present invention provides a process for preparing a solid pharmaceutical composition comprising Azilsartan or its salt and one or more pharmaceutically acceptable excipient(s); wherein the said composition further comprises a diuretic; specifically the diuretic includes thiazide diuretics; more specifically the thiazide diuretic is Chlorthalidone.

### BRIEF DESCRIPTION OF THE DRAWINGS

FIG. 1 represents dissolution data for Example 01, Table No. 03

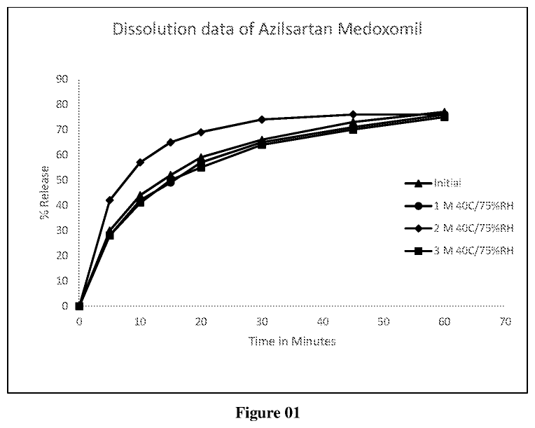

FIG. 2 represents dissolution data for Example 02, Table No. 06

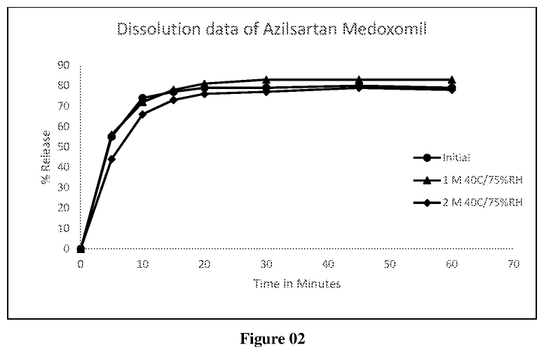

FIG. 3 represents dissolution data for Example 03, Table No. 09

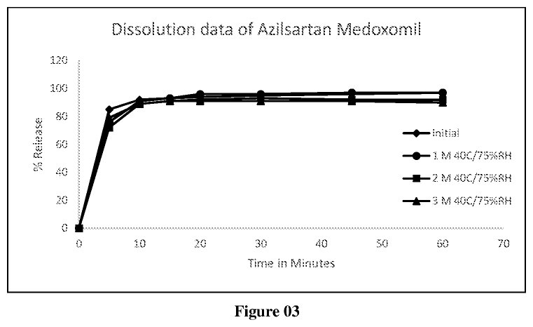

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the definitions herein, may utilize the present invention to its fullest extent. The following specific embodiments are to be construed as merely illustrative, and not limitative of the remainder of the disclosure in any way whatsoever.

Unless otherwise defined, all the terms used herein, including the technical and scientific terms, have the meaning as that generally understood by one of ordinary skill in the art to which the present invention relates.

### Definitions

The general terms used hereinbefore and hereinafter preferably have the following meanings within the context of this disclosure, unless otherwise indicated. Thus, the definitions of the general terms as used in the context of the present invention are provided herein below:

The terms “a”, “an” and “the” refers to “one or more” when used in the subject specification, including the claims. Thus, for example, reference to “a disease” or “a condition” includes a plurality of diseases or disorders.

It should also be noted that the term “or” is generally employed in its sense including “and/or” unless the content clearly dictates otherwise.

As used herein the term “pharmaceutically acceptable” is meant that the carrier, diluent, excipient(s), and/or salt must be compatible with the other ingredients of the formulation, and not deleterious to the recipient thereof. “Pharmaceutically acceptable” also means that the compositions or dosage forms are within the scope of sound medical judgment, suitable for use for an animal or human without excessive toxicity, irritation, allergic response, or other problem or complication, commensurate with a reasonable benefit/risk ratio.

The term “pharmaceutical combination” or “combination” as used herein means the combined administration of the therapeutic agents. In the context of the present invention, the therapeutic agents include an angiotensin II receptor blocker (ARB) and a diuretic that can be administered independently at the same time or separately within time intervals such that these time intervals allow the combination partners to exhibit a synergistic effect.

As used herein, the term “pH” is a measure of hydrogen ion concentration, as commonly used in the art. Customarily, the pH provides a measure on a scale from 0 to 14 of the acidity or alkalinity of a solution.

As used herein, the term “about” refers to a range of values ±10% of a specified value.

As used in this disclosure, “API” or “API-01” or “API-02” is an abbreviation for active pharmaceutical ingredient. API means, any substance or compound to be used in the manufacture of a drug product. Such substances furnish pharmacological activity or other direct effect in the diagnosis, cure, mitigation, treatment or prevention of disease or to affect the structure and function of the body. In the context of the present invention, API-01 and API-02 refer to the active pharmaceutical ingredient “Azilsartan or its salt” and “thiazide diuretic such as Chlorthalidone” respectively.

Within the context of the present invention and as used herein, unless indicated otherwise, references to total weight of the pharmaceutical composition refers to the total weight of the active agent(s) and pharmaceutically acceptable excipient(s).

Within the context of the present invention and as used herein the term “subject” refers to an animal, preferably a mammal, and most preferably a human. In the context of the present invention, the term “mammal” is used interchangeably with the term “patient” or “subject”. In the context of the present invention, the phrase “a subject in need thereof” means a subject (patient) in need of the treatment of a disease or disorder for which drug substance is used.

Within the context of the present invention and as used herein the term ‘diluent’ refers to an agent used as filler in order to achieve the desired composition volume or weight. The diluent may be present in the pharmaceutical composition in the form of a single compound or in the form of a mixture of compounds. Diluents are often added to tablet formulations to provide better tablet properties such as to improve cohesion, to allow direct compression manufacturing, to enhance flow and to adjust weight of tablet as per die capacity. Diluents are generally classified into three categories namely organic, inorganic and co-processed diluents. The organic diluents include but are not limited to, lactose such as α-lactose monohydrate, spray dried lactose and anhydrous lactose, starch such as potato starch, corn starch or maize starch, and pregelatinized starch, icing sugar with starch, sucrose, mannitol, sorbitol, cellulose such as powdered cellulose and microcrystalline cellulose. The inorganic diluents include but are not limited to calcium phosphates such as anhydrous dibasic calcium phosphate, dibasic calcium phosphate and tribasic calcium phosphate. Some of the insoluble diluents include but are not limited to starch, powdered cellulose, microcrystalline cellulose, calcium phosphate and the like. Some of the soluble diluents include but are not limited to lactose, sucrose, mannitol, sorbitol and the like.

Binders are dry powders or liquid which are added during granulation process to promote granules and cohesiveness. Binders are, but not limited to, cellulose and its derivatives including, ethyl cellulose, hydroxypropyl cellulose, hydroxypropyl methylcellulose (hypromellose), methylcellulose and hydroxyethyl cellulose, carboxymethyl cellulose, gelatin, liquid glucose, corn starch or maize starch, pregelatinized starch, hydrocolloids, sugars, polyvinyl pyrrolidone, sodium alginate, acacia, alginic acid, tragacanth, xanthan, used either alone or combinations thereof.

Disintegrant as used in herein refers to any material that facilitates the break-up of a tablet prepared from the composition when placed in contact with an aqueous medium. Suitable disintegrants include, but are not limited to, crospovidone, sodium starch glycolate, hydroxypropyl starch, microcrystalline cellulose, carboxymethylcellulose sodium or calcium, croscarmellose sodium, pregelatinized starch, polacrilin potassium, low-substituted hydroxypropylcellulose, sodium or calcium alginate, agar, guar gum, chitosan, alginic acid and mixtures thereof.

Glidants improve the flowability of the composition. Exemplary glidants are, but not limited to, fumed silica (colloidal silicon dioxide), colloidal silica, powdered cellulose, talc, tribasic calcium phosphate, magnesium stearate, magnesium carbonate, mixtures thereof and the like.

Lubricants are added in small quantities to tablet formulations to improve certain processing characteristics. The role of the lubricants is to ensure that tablet formation and ejection can occur with low friction between the tablet ingredients and the die walls of the tableting machine. Lubricant prevents sticking to punch faces and enhances product flow by reducing interparticulate friction. The lubricant may be present in the pharmaceutical composition in the form of a single compound or in the form of a mixture of compounds. Lubricants are, but not limited to sodium oleate, sodium stearate, sodium benzoate, sodium chloride, stearic acid, sodium stearyl fumarate, calcium stearate, magnesium stearate, magnesium lauryl sulfate, sodium stearyl fumarate, sucrose esters or fatty acid, zinc, polyethylene glycol, talc, mixtures thereof and the like.

Generally, suitable surfactants include anionic, cationic, and nonionic surfactants, or combinations thereof. In one aspect, such surfactants are nonionic and/or anionic surfactants. In one aspect, the concentration of surfactant is about 0.1% w/w to about 2% w/w;

One or more of these excipient(s) can be selected and used by the artisan having regard to the particular desired properties of the solid dosage form. The amount of each type of excipient employed, e.g. diluent, binder, disintegrant, pH adjusting agent, glidant and lubricant may vary within ranges conventional in the art.

Coating materials are polymeric or non-polymeric, but not limited to, sugars, hydroxypropyl methylcellulose (hypromellose), hydroxypropyl cellulose, methylcellulose, ethylcellulose, polyvinyl alcohol, sodium carboxylmethylcellulose, coatings based on methacrylic acid and its esters, such as Eudragit®, mixtures thereof and the like. As alternatives for the above coating materials, sometimes pre-formulated coating products such as those sold as OPADRY™ will be used, for example Opadry White or Opadry Green. The products sold in a solid form require only mixing with a liquid before use. Alternatively, film-forming agents may be applied as powders, using suitable powder coating equipment known in the art.

The vehicle used to prepare the dispersion may be selected from water or its mixture with other organic solvent such as ethanol, methanol, isopropyl alcohol and ether.

Suitable pharmaceutical compositions include, but are not limited to, capsules, tablets, granules, powders and unit dose pockets. Preferably the oral pharmaceutical composition is a tablet.

In an embodiment, the present invention provides a stable solid pharmaceutical composition comprising Azilsartan or its salt and one or more pharmaceutically acceptable excipient(s) thereof; optionally the composition comprises one or more diuretics. The invention further relates to a method of treating hypertension in a subject in need thereof, wherein the said composition is administered orally.

In another embodiment, the present invention relates to a stable solid pharmaceutical composition comprising Azilsartan or its salt and one or more pharmaceutically acceptable excipient(s); wherein one or more pharmaceutically acceptable excipient(s) present in the oral dosage form include but are not limited to fillers, diluents, binders, disintegrants, coating agents, glidants, surfactants, pH modifiers or pH adjusting agents, lubricants, vehicle, and the like.

In another embodiment, the concentration of surfactant in the composition is present in the range from about 0.1% w/w to about 2% w/w.

In another embodiment, the concentration of pH adjusting agent(s) in the composition is present in the range from about 0.1% w/w to about 4% w/w.

In yet another embodiment, the present invention provides stable solid pharmaceutical composition comprising Azilsartan kamedoxomil and one or more pharmaceutically acceptable excipient(s), wherein pH adjusting agent include but not limited to fumaric acid and sodium hydroxide; wherein the ratio of fumaric acid and sodium hydroxide is about 1:0.2 to about 1:2, specifically about 1:0.4 to about 1:1; more specifically about 1:0.6 to about 1:0.7.

In another embodiment, the present invention provides stable solid pharmaceutical composition comprising Azilsartan kamedoxomil and one or more pharmaceutically acceptable excipient(s), wherein ratio of sodium hydroxide to crospovidone from extragranular material is in the range from about 1:1 to about 1:20.

In another embodiment, the present invention provides use of compound of formula I alone or in combination with compound of formula II for the treatment of hypertension in a subject in need thereof.

In another embodiment, the present invention provides stable solid pharmaceutical composition comprising Azilsartan kamedoxomil and one or more pharmaceutically acceptable excipient(s), wherein 1% w/v solution of acid and base combination results in pH above 5, specifically in the range from 5.1 to 7.0.

In another embodiment, the present invention provides stable solid pharmaceutical composition comprising Azilsartan kamedoxomil and one or more pharmaceutically acceptable excipient(s), wherein the composition further comprises use of one or more diuretics. In the context of the present invention, the pH of a solution or suspension is obtained by dissolving or suspending the pH adjusting agent in water at 25° C. to 40° C. at a concentration of about 0.1% w/v to 3% w/v is ≥5. Preferably the pH is of a solution or suspension obtained by dissolving or suspending the pH adjusting agent in water at a concentration of about 1% w/v to 3% w/v which is in the range from about 5.1 to about 7.0; wherein said composition provides stable dosage for oral administration. Specifically, the pH of a solution or suspension is obtained by dissolving or suspending the pH adjusting agent in water at 25° C. at a concentration of about 1% w/v to 3% w/v which is in the range from about 5.1 to about 7.0.

In another embodiment, the present invention provides stable solid pharmaceutical composition comprising combination of Azilsartan kamedoxomil and Chlorthalidone and one or more pharmaceutically acceptable excipient(s), wherein the composition can be administered orally for the treatment of lowering the blood pressure in a subject in need thereof.

In an embodiment, the present invention provides a method of stabilizing a compound of formula I or its salt optionally in combination with a diuretic as presented in compound of formula II, and a pH modifying agent. According to stabilizing method of the present invention, compound of formula I or its salt and optionally a diuretic in a solid preparation is significantly stabilized when the pH of a solution or suspension obtained by dissolving or suspending the pH adjusting agent in water at a concentration of about 0.1% w/v to 3% w/v is in the range from about 5.1 to about 7.0; wherein said composition provides stable dosage for oral administration. Specifically, the pH of a solution or suspension obtained by dissolving or suspending the pH adjusting agent in water at 25° C. at a concentration of about 1% w/v is in the range from about 5.1 to about 7.0.

In this context, 1% w/v solution or dispersion of acid and base combination are used in the composition, giving pH range from about 5.1 to about 7.0. When Azilsartan kamedoxomil and Chlorthalidone were granulated separately and dried, mixed together, lubricated, compressed into tablets and coated, the impurity levels of Azilsartan kamedoxomil were found to be low.

In another embodiment, the present invention is administered by oral route.

In another embodiment, the present composition provides stable solid dosage form, wherein the solid dosage form includes tablets, capsules, powder, pills, coated tablets, preferably tablets.

In a further embodiment, the present invention provides stable solid pharmaceutical composition comprising:
(a) about 6% w/w to about 30% w/w of Azilsartan kamedoxomil;
(b) about 1% w/w to about 10% w/w of disintegrant(s);
(c) about 0.1% w/w to about 3% w/w of pH modifier(s);
(d) about 0.5% w/w to about 5% w/w to about of binder(s);
(e) optionally, about 1% w/w to about 10% w/w of diuretic(s); and
(f) one or more pharmaceutically acceptable excipient(s);
   wherein said composition is used in the treatment of hypertension in a subject in need thereof and said composition provides tablet dosage form for oral administration.

In another embodiment, the preferred diuretics in the present invention are thiazide diuretics like, Chlorothiazide, Chlorthalidone, Hydrochlorothiazide, Indapamide, Metolazone; preferred diuretic as per present invention is Chlorthalidone.

In another embodiment, the preferred diuretic in the present invention is Chlorthalidone.

In a further embodiment, the present invention provides stable solid pharmaceutical composition comprising:
(a) about 6% w/w to about 30% w/w of Azilsartan kamedoxomil;
(b) about 1% w/w to about 10% w/w of disintegrant(s);
(c) about 0.1% w/w to about 3% w/w of pH modifier(s);
(d) about 0.5% w/w to about 5% w/w to about of binder(s);
(e) optionally, about 1% w/w to about 10% w/w of thiazide diuretic(s); and
(f) one or more pharmaceutically acceptable excipient(s);
   wherein said composition is used in the treatment of hypertension in a subject in need thereof and said composition provides tablet dosage form for oral administration.

In further embodiment, the present invention provides process for preparing a stable solid composition comprising:
(a) about 6% w/w to about 30% w/w of Azilsartan kamedoxomil;
(b) about 1% w/w to about 10% w/w of disintegrant(s);
(c) about 0.1% w/w to about 3% w/w of pH modifier(s);
(d) about 0.5% w/w to about 5% w/w to about of binder(s);
(e) optionally, about 1% w/w to about 10% w/w of Chlorthalidone; and
(f) one or more pharmaceutically acceptable excipient(s);
   wherein, the said composition is used in the treatment of hypertension in a subject in need thereof and said composition provides tablet dosage form for oral administration.

In a further embodiment, the present invention provides a stable solid pharmaceutical composition comprising Azilsartan kamedoxomil; one or more pH adjusting agents and one or more pharmaceutically acceptable excipient(s) thereof; wherein the composition contains pH adjusting agent in 1% w/v solution of acid and base combination resulting in pH range from 5.1 to 7.0.

In further embodiment, the present composition provides use of fumaric acid (acid) and sodium hydroxide (base), which results in complete dissolution and detection of low level of impurities. Solution of about 1% w/v of acid and base combination is used in the composition, which gives pH range from about 5.1 to 7.0.

In a further embodiment, the present invention provides a process for preparation of stable solid pharmaceutical composition comprising Azilsartan or its salt and one or more pharmaceutically acceptable excipient(s) thereof; optionally the composition comprises one or more diuretics.

In further embodiment, the present invention provides process for preparing solid pharmaceutical composition comprising Azilsartan or its salt and one or more pharmaceutically acceptable excipient(s) thereof; wherein the process comprises the steps of:
a) co-sifting intragranular material (API-01, diluent and disintegrant) through a sieve, adding material to fluidized bed processor bowl and mixing for 5 minutes;
b) dissolving binder and pH modifier in purified water under continuous stirring to constitute binder solution;
c) granulating material of step (a) with the binder solution of step (b) in fluidized bed processor followed by drying;
d) drying granules being passed through a sieve;
e) mixing dried granules and sifted extragranular material (diluent, disintegrant and surfactant) for 10 minutes in a conta blender;
f) passing lubricant through a sieve and lubricating the blend of step (e) for 03 minutes in conta blender;
g) compressing lubricated blend of step (f) into tablets in rotary compression machine; and
h) optionally coating the tablets of step (g) with a solution comprising one or more coating agent and vehicle.

In further embodiment, the present invention provides a process for preparing solid pharmaceutical composition comprising Azilsartan or its salt and one or more pharmaceutically acceptable excipient(s) thereof in combination with a diuretic; wherein the process comprises the steps of:
a) co-sifting intragranular material (API-01) and API-02) and/or separately sifted API-02; diluent; and disintegrant through a sieve, adding material to fluidized bed processor bowl and mixing for 5 minutes;
b) dissolving binder and pH modifier in purified water under continuous stirring to constitute binder solution;
c) granulating material of step (a) with the binder solution of step (b) in fluidized bed processor followed by drying;
d) drying granules being passed through a sieve;
e) mixing dried granules and sifted extragranular material (diluent, disintegrant and surfactant) for 10 minutes in a conta blender;
f) passing lubricant through a sieve and lubricating the blend of step (e) for 03 minutes in conta blender;
g) compressing lubricated blend of step (f) into tablets in rotary compression machine; and
h) optionally coating the tablets of step (g) with a solution comprising one or more coating agent and vehicle.

In further embodiment, the present invention provides a process for preparing solid pharmaceutical composition comprising Azilsartan or its salt and one or more pharmaceutically acceptable excipient(s) thereof in combination with a diuretic; wherein the process comprises the steps of:
a) co-sifting granulating material (API-01; diluent; and disintegrant) through a sieve, adding material to fluidized bed processor bowl and mixing for 5 minutes;
b) co-sifting granulating material (API-02; diluent; and disintegrant) through a sieve, adding material to fluidized bed processor bowl and mixing for 5 minutes;
c) dispersing/dissolving binder and pH modifier in purified water under continuous stirring to constitute binder solution;
d) granulating material of step (a) and step (b) with the binder solution of step (c) in fluidized bed processor separately followed by proper drying and mixing; e) drying granules passed through a sieve;
f) mixing dried granules and sifted extragranular material (diluent, disintegrant and surfactant) for 10 minutes in a conta blender;
g) passing lubricant through a sieve and lubricating blend of step (f) for 03 minutes in conta blender;
h) compressing lubricated blend of step (g) into tablets in rotary compression machine; and
i) optionally coating the tablets of step (h) with solution comprising one or more coating agent and vehicle.

In separate embodiments, the present invention provides a process for preparing stable pharmaceutical composition comprising Azilsartan or its salt and one or more pharmaceutically acceptable excipient(s) thereof; wherein the process comprises steps of:
a) dispensing all the excipient(s) accurately;
b) sifting of intragranular material (API-01, mannitol, microcrystalline cellulose and crospovidone) through a sieve. Further, adding all the intragranular material to fluidized bed processor bowl and mixing for 10 minutes;
c) preparing binder solution in two steps; firstly dissolving sodium hydroxide in purified water followed by fumaric acid and stirring continued till all material get dissolved; and secondly, adding hydroxypropyl cellulose or PVP K30 to purified water under continuous stirring till material gets dissolved;
d) spraying solution obtained from first part of step (c) on the material of step (b) and further, spraying of binder solution obtained from second part of step (c) on the same material in fluidized bed processor;
e) drying of the material in fluidized bed processor to achieve desired LOD;
f) sifting of dried granules through a sieve;
g) weighing of extragranular material (crospovidone) according to yield of dried granules and passing through a sieve;
h) mixing of the dried granules obtained from step (f) and material obtained from step (g) for 10 minutes in conta blender;
i) sifting of magnesium stearate through a sieve;
j) lubricating the blend obtained from step (h) with magnesium stearate for 3 minutes in conta blender;
k) compressing the blend obtained from step (j) in rotary compression machine and obtaining tablets dosage form; and
l) optionally, coating the tablets of step (k) with the suitable coating agent (Opadry).

In another embodiment, the present invention provides a process for preparing stable pharmaceutical composition comprising Azilsartan or its salt and one or more pharmaceutically acceptable excipient(s) thereof in combination with Chlorthalidone; wherein the process comprises steps of:
A. Process for preparation of Chlorthalidone Granulation (Part 1):
   a) dispensing all the excipient(s) accurately;
   b) co-sifting of intragranular material (Chlorthalidone, microcrystalline cellulose, part quantity of mannitol and crospovidone) through a sieve and mixing into fluidized bed processor (FBP) bowl;
   c) dissolving PVP K 30 into purified water under continuous stirring;
   d) granulating the material of step (b) with material of step (c) followed by drying of the granules;
   e) passing the dried granules through a sieve.
B. Process for preparation of Azilsartan medoxomil Granulation (Part 2):
   f) dispensing all the excipient(s) accurately;
   g) co-shifting of intragranular material (API, 80% of mannitol) through a sieve; and sifting of microcrystalline cellulose through a sieve;
   h) sifting of remaining quantity of mannitol through a sieve and adding to fluidized bed processor bowl and mixing for 5 minutes;
   i) adding the material of step (g) to step (h) and mixing for 5 minutes in FBP bowl;
   j) preparing binder solution in two steps; firstly dissolving sodium hydroxide in purified water followed by fumaric acid and stirring continued till all material get dissolved; and secondly, adding hydroxypropyl cellulose to purified water under continuous stirring till material gets dissolved;
   k) spraying the solution obtained in part one of step (j) on material in fluidized bed processor and further spraying binder solution obtained from second part of step (j) on same material in fluidized bed processor;
   l) drying the material in fluidized bed processor to achieve desired LOD;
   m) sifting of the dried granules through a sieve.
C. Blending with both API granules (Part 3):
   n) mixing of Azilsartan medoxomil granules with Chlorthalidone granules for 20 minutes in conta blender;
   o) sifting of crospovidone through a sieve and mixed with material obtained from step (n) for 5 minutes;
   p) sifting of magnesium stearate through a sieve;
   q) lubricating the blend with magnesium stearate for 5 minutes in conta blender;
   r) compressing the blend obtained from step (q) into tablets in rotary compression machine;
   s) preparing coating solution of Opadry in purified water and coating the compressed tablets.

In separate embodiment, the present invention provides a method of treating a patient by administering a stable solid pharmaceutical composition comprising Azilsartan or its salt and one or more pharmaceutically acceptable excipient(s) thereof; optionally the composition comprising one or more diuretics; wherein the diuretic is Chlorthalidone.

Examples of the disintegrant include low substituted hydroxypropylcellulose, carmellose, carboxy-methyl-starch sodium, cross povidone, amino acid, starch, cornstarch, carboxymethylcellulose, calcium carboxymethylcellulose, sodium carboxymethyl starch, carmellose sodium, carmellose calcium, croscarmellose sodium, hydroxypropylstarch, sodium carboxymethyl starch and the like.

Examples of the binders include povidone, dextrin, hydroxypropylcellulose, hydroxypropyl methylcellulose, methyl cellulose, polyvinyl alcohol, carboxymethylcellulose, pregelatinized starch, cellulose (e.g., microcrystalline cellulose), gelatin, starch, gum arabic powder, tragacanth, sodium alginate, pullulan, glycerol and powdered acacia.

Examples of the fillers or diluents include mannitol (eg. D-mannitol); white sugar (including purified white sugar); sorbitol (e.g., D-sorbitol), erythritol (e.g., D-erythritol), sucrose; sodium hydrogencarbonate; corn starch; potato starch; wheat starch; rice starch; partly alpha-sized starch; crystalline cellulose; light anhydrous silicic acid; precipitated calcium carbonate; calcium silicate; sodium hydrogen carbonate, calcium phosphate, calcium sulfate, calcium carbonate, precipitated calcium carbonate, calcium silicate and the like.

Examples of the lubricants include hardened oils, hydrogenated-castor oil, magnesium stearate, calcium stearate, glyceride behenate; sodium stearyl fumarate; stearic acid, talc (purified talc), sucrose esters of fatty acid, and the like.

Examples of the surfactant include sodium lauryl sulfate, polysorbate 80, polysorbate 20, polyoxyethylene(160); polyoxypropylene(30) glycol and the like.

Examples of the diuretic include xanthine derivatives (e.g., theobromine sodium salicylate, theobromine calcium salicylate, etc.), thiazide preparations (e.g., ethiazide, cyclopenthiazide, trichloromethiazide, hydrochlorothiazide, hydroflumethiazide, benzylhydrochlorothiazide, penflutizide, polythiazide, methyclothiazide, etc.), antialdosterone preparations (e.g., spironolactone, triamterene, etc.), carbonic anhydrase inhibitors (e.g., acetazolamide, etc.), chlorobenzenesulfonamide agents (e.g., chlortalidone, mefruside, indapamide, etc.), azosemide, isosorbide, ethacrynic acid, piretanide, bumetanide, furosemide and the like. In the present invention, preferable diuretic is thiazide diuretic, more specifically Chlorthalidone.

The above-mentioned composition preferably contains compound (I) (preferably (5-methyl-2-oxo-1,3-dioxol-4-yl)methyl 2-ethoxy-1-f{[2′-(5-oxo-4,5-dihydro-1,2,4-oxadiazol-3-yl)biphenyl-4-yl]met-hyl}-1H-benzimidazole-7-carboxylate potassium salt); a pH adjusting agent (preferably fumaric acid and sodium hydroxide); a surfactant (preferably sodium lauryl sulphate); a diluent (preferably mannitol and crystalline cellulose); and a binder (preferably hydroxypropylcellulose).

### EXAMPLES

### Example 01

TABLE 01
Azilsartan composition

| Ingredient | mg/tab |
|---|---|
| Azilsartan kamedoxomil | 85.360 |
| Mannitol | 208.14 |
| Croscarmellose sodium | 8.000 |
| Hydroxypropyl Cellulose | 8.000 |
| Purified water | Q.S |
| Micro crystalline cellulose | 30.000 |
| Croscarmellose sodium | 7.000 |
| Magnesium Stearate | 3.500 |
| Total | 350.00 |

### Manufacturing Procedure of Example 01:

1. The active pharmaceutical ingredient (i.e. Azilsartan kamedoxomil), mannitol, and croscarmellose sodium were granulated in fluidized bed granulator by using binder solution of hydroxypropyl cellulose and purified water. The obtained granules were dried in fluidized bed processor to achieve desired LOD. The dried granules were passed through 18 mesh sieve;
2. the extragranular materials (micro crystalline cellulose and croscarmellose sodium) were weighed according to the yield of dried granules and passed through a 30 mesh sieve. The dried granules and extragranular material were mixed in conta blender;
3. the blend obtained in step 2 was lubricated with magnesium stearate in conta blender; and
4. the lubricated blend was compressed into tablets in rotary compression machine.

TABLE 02
Stability Data for Example 01:
Impurity data

| | Stage | | | | |
|---|---|---|---|---|---|
| | | 1 M 40° | 2 M 40° | 3 M 40° | 6 M 40° |
| | | C./75% | C./75% | C./75% | C./75% |
| | Initial | RH | RH | RH | RH |
| Total degradation | 5.62 | 7.37 | 8.25 | 8.91 | 11.74 |
| product for | | | | | |
| Azilsartan | | | | | |
| medoxomil in % | | | | | |

TABLE 03
Dissolution Data for Example 01:
Dissolution data
Dissolution Condition - Phosphate Buffer, pH
7.8 (deaerated), II (Paddle), 50 rpm, 900 ml

| | Stage | | | |
|---|---|---|---|---|
| Time | | 1 M 40° | 2 M 40° | 3 M 40° |
| Points in | Initial | C./75% RH | C./75% RH | C./75% RH |
| minutes | [% Drug Release and (% RSD)] | | | |
| 5 | 30 (5.8) | 28 (5.4) | 42 (2.3) | 28 (2.3) |
| 10 | 44 (2.6) | 42 (4.1) | 57 (1.7) | 41 (2.7) |
| 15 | 52 (1.1) | 49 (9.4) | 65 (1.0) | 50 (2.3) |
| 20 | 59 (1.0) | 57 (3.0) | 69 (1.2) | 55 (2.2) |
| 30 | 66 (0.9) | 65 (4.5) | 74 (0.7) | 64 (2.4) |
| 45 | 73 (2.1) | 71 (5.4) | 76 (0.8) | 70 (3.8) |
| 60 | 77 (0.7) | 76 (2.0) | 76 (0.5) | 75 (1.6) |

### Example 02

TABLE 04
Composition

| Ingredient | mg/tab |
|---|---|
| Azilsartan kamedoxomil | 85.360 |
| Mannitol | 194.90 |
| Croscarmellose sodium | 9.000 |
| Hydrochloric Acid | 0.937 |
| Hydroxy propyl cellulose | 6.000 |
| Purified water | Q.S |
| Micro crystalline cellulose | 36.000 |
| Sodium Lauryl Sulphate | 3.500 |
| Croscarmellose sodium | 9.000 |
| Magnesium Stearate | 5.300 |
| Total | 350.00 |

### Manufacturing Procedure of Example 02:

1. The active pharmaceutical ingredient (i.e. Azilsartan kamedoxomil), mannitol, and croscarmellose sodium were granulated in fluidized bed granulator by using binder solution of hydroxypropyl cellulose and purified water. The obtained granules were dried in fluidized bed processor to achieve desired LOD. The dried granules were passed through a 18 mesh sieve;
2. the extragranular materials (microcrystalline cellulose, sodium lauryl sulphate and croscarmellose sodium) were weighed according to the yield of dried granules and passed through a 30 mesh sieve. The dried granules and extragranular material were mixed in conta blender;
3. the blend obtained in Step 2 was lubricated with magnesium stearate for conta blender; and
4. the lubricated blend was compressed into tablets in rotary compression machine.

TABLE 05
Stability results of Example 02
Impurity data

| | Stage | | | |
|---|---|---|---|---|
| | | 1 M 40° | 2 M 40° | 3 M 40° |
| | Initial | C./75% RH | C./75% RH | C./75% RH |
| Total degradation | 4.93 | 3.80 | 4.71 | 5.60 |
| product for | | | | |
| Azilsartan | | | | |
| medoxomil in % | | | | |

TABLE 06
Dissolution Data for Example 02:
Dissolution data
Dissolution Condition - Phosphate Buffer, pH
7.8 (deaerated), II (Paddle), 50 rpm, 900 ml

| Stage | | 1 M | 2 M |
|---|---|---|---|
| Time Points in | Initial | 40° C./75% RH | 40° C./75% RH |
| minutes | [% Drug Release and (% RSD)] | | |
| 5 | 55 (22.5) | 56 (1.4) | 44 (28.8) |
| 10 | 74 (1.1) | 72 (0.6) | 66 (7.8) |
| 15 | 77 (1.1) | 78 (1.5) | 73 (2.4) |
| 20 | 79 (1.3) | 81 (1.1) | 76 (1.3) |
| 30 | 79 (1.1) | 83 (0.7) | 77 (1.1) |
| 45 | 80 (1.3) | 83 (0.5) | 79 (0.7) |
| 60 | 79 (1.1) | 83 (1.40) | 78 (0.8) |

### Example 03

TABLE 07
Composition

| | |
|---|---|
| Azilsartan kamedoxomil | 85.360 |
| Mannitol | 175.01 |
| Croscarmellose sodium | 9.000 |
| Fumaric acid | 4.000 |
| Sodium hydroxide | 2.680 |
| Hydroxy propyl cellulose | 6.000 |
| Purified water | Q.S |
| Micro crystalline cellulose | 36.000 |
| Croscarmellose sodium | 7.000 |
| Magnesium Stearate | 4.950 |
| Total | 330.00 |

### Manufacturing Procedure of 2Example 03:

1. The active pharmaceutical ingredient (i.e. Azilsartan kamedoxomil), mannitol, and croscarmellose sodium were granulated in fluidized bed granulator by spraying an aqueous solution hydroxypropyl cellulose and purified water along with solution of fumaric acid and sodium hydroxide. The obtained granules were dried in fluidized bed processor to achieve desired LOD. The dried granules were passed through a 18 mesh sieve;
2. the extragranular materials (microcrystalline cellulose, and croscarmellose sodium) were weighed according to the yield of dried granules and passed through a 30 mesh sieve. The dried granules and extragranular material were mixed in conta blender;
3. the blend obtained in step 2 was lubricated with magnesium stearate in conta blender; and
4. the lubricated blend was compressed into tablets in rotary compression machine.

TABLE 08
Stability results of Example 03
Impurity data

| | | 1 M | 6 M |
|---|---|---|---|
| Stage | Initial | 40° C./75% RH | 400 C./75% RH |
| Total degradation product | 2.28 | 2.19 | 2.66 |
| for Azilsartan medoxomil | | | |
| in % | | | |

TABLE 09
Dissolution Data for Example 03:
Dissolution data
Dissolution Condition - Phosphate Buffer, pH
7.8 (deaerated), II (Paddle), 50 rpm, 900 ml

| Stage | | 1 M | 6 M |
|---|---|---|---|
| Time Points in | Initial | 40° C./75% RH | 400 C./75% RH |
| minutes | [% Drug Release and (% RSD)] | | |
| 5 | 85 (3.6) | 76 (4.7) | 72 (5.5) |
| 10 | 92 (0.8) | 91 (1.1) | 89 (1.9) |
| 15 | 93 (0.4) | 93 (1.6) | 92 (1.0) |
| 20 | 94 (1.6) | 96 (1.3) | 93 (1.6) |
| 30 | 95 (1.3) | 96 (2.1) | 93 (0.8) |
| 45 | 96 (1.5) | 97 (1.9) | 95 (1.3) |
| 60 | 97 (1.4) | 97 (1.8) | 95 (1.1) |

Observation: The composition with fumaric acid (acid) and sodium hydroxide (base) exhibited low level of impurities and complete dissolution. 1% w/v solution/dispersion of acid and base combination used in the composition resulted in pH range from 5.1 to 7.0.

### Example 4

TABLE 10
Composition

| Ingredient | mg/tab |
|---|---|
| Azilsartan kamedoxomil | 42.680 |
| Chlorthalidone | 25.000 |
| Mannitol | 219.04 |
| Crospovidone | 8.000 |
| Fumaric acid | 4.000 |
| Sodium hydroxide | 2.680 |
| Hydroxy propyl cellulose | 8.000 |
| Purified water | Q.S |
| Micro crystalline cellulose | 30.000 |
| Crospovidone | 7.000 |
| Magnesium Stearate | 3.600 |
| Total weight of uncoated tablets | 350.00 |
| Opadry | 10.000 |
| Coating polymer/system/material | |
| Purified water | Q.S |
| Total weight of coated tablets | 360.000 |

### Manufacturing Procedure of Example 04:

1. All the intragranular materials [Active pharmaceutical ingredients (i.e. Azilsartan kamedoxomil and Chlorthalidone), mannitol, and crospovidone] were loaded into fluidized bed granulator;
2. prepared granulating/binder solution as follows:
   A. hydroxypropyl cellulose is added to purified water under continuous stirring. Stirring is continued till material dissolved;
   B. fumaric acid and sodium hydroxide is added to purified water one by one and stirred to form clear solution.
   C. solution of step B is added to solution of step A;
3. the solution obtained in step 2C was sprayed on material of step 1 in fluidized bed processor;
4. the obtained granules were dried in fluidized bed processor to achieve desired LOD. The dried granules were passed through a 16 mesh sieve;
5. the extragranular materials (microcrystalline cellulose, and crosspovidone) were weighed according to the yield of dried granules and passed through a 30 mesh sieve and mixed with material obtained in step 4;
6. the obtained material was lubricated with magnesium stearate;
7. the lubricated blend was compressed into tablets in rotary compression machine;
8. the Opadry was dispersed in purified water under continuous stirring and stirred for 45 minutes.
9. the compressed tablets obtained from step 7 were coated with Opadry.

TABLE 11
Stability results of Example 04
Impurity Data

| | Azilsartan medoxomil | | | | Chlorthalidone | | | |
|---|---|---|---|---|---|---|---|---|
| | | 1 M | 2 M | 3 M | | 1 M | 2 M | 3 M |
| | | 40°/75% | 40°/75% | 40°/75% | | 40°/75% | 40°/75% | 40°/75% |
| Stage | Initial | RH | RH | RH | Initial | RH | RH | RH |

Azilsartan medoxomil Impurity

| | | | | | |
|---|---|---|---|---|---|
| Total | 6.28 | 5.94 | 7.48 | 8.10 | NA |
| degradation | | | | | |
| product for | | | | | |
| Azilsartan | | | | | |
| medoxomil | | | | | |
| in % | | | | | |

Chlorthalidone Impurity

| | | | | | |
|---|---|---|---|---|---|
| Total | NA | 0.16 | 0.13 | 0.19 | 0.21 |
| degradation | | | | | |
| product for | | | | | |
| Chlorthalidone | | | | | |
| in % | | | | | |

### Example 5

TABLE 12
Chlorthalidone Granules Composition

| | | |
|---|---|---|
| Sr. | | |
| No. | Ingredient | mg/tab |

Intragranular Material

| | | |
|---|---|---|
| 1 | Chlorthalidone | 25.000 |
| 2 | Mannitol | 85.620 |
| 3 | Microcrystalline cellulose | 9.000 |
| 4 | Crospovidone | 8.000 |

Binder

| | | |
|---|---|---|
| 5 | Hydroxy propyl cellulose | 5.400 |
| 6 | Purified water | Q.S |

| | |
|---|---|
| Total | 133.020 |

TABLE 13
Azilsartan medoxomil granules composition

| | | |
|---|---|---|
| Sr. | | |
| No. | Ingredient | mg/tab |

Intragranular Material

| | | |
|---|---|---|
| 1 | Azilsartan kamedoxomil | 42.680 |
| 2 | Mannitol | 80.320 |
| 3 | Microcrystalline cellulose | 9.000 |
| 4 | Crospovidone | 8.000 |

Binder

| | | |
|---|---|---|
| 5 | Fumaric acid | 4.000 |
| 6 | Sodium hydroxide | 2.680 |
| 7 | Hydroxy propyl cellulose | 5.400 |
| 8 | Purified water | Q.S |

TABLE 14
Lubricated blend component

| Sr. | | |
|---|---|---|
| No. | Ingredient | mg/tab |
| 1 | Azilsartan granules | 152.080 |
| 2 | Chlorthalidone granules | 133.020 |
| 3 | Micro crystalline cellulose | 36.000 |
| 4 | Crospovidone | 4.000 |
| 5 | Magnesium Stearate | 4.900 |
| Total weight of uncoated tablets | | 330.000 |
| 6 | Opadry | 10.000 |
| 7 | Purified water | Q.S |

| | |
|---|---|
| Total weight of coated tablets | 340.000 |

### Manufacturing Procedure of Example 05:

A. Chlorthalidone Granulation:
   1. The intragranular materials (Chlorthalidone, microcrystalline cellulose, mannitol and crospovidone) were granulated by using binding solution of hydroxypropyl cellulose with purified water; and
   2. the material obtained in step 1 was then dried and passed through 18 mesh sieve.
B. Azilsartan medoxomil Granulation:
   1. The intragranular materials (Azilsartan, mannitol, microcrystalline cellulose and crospovidone) were granulated by using binder solution of sodium hydroxide and purified water followed by fumaric acid in fluidized bed processor; and
   2. the material obtained by step 1 was then dried and passed through 18 mesh sieve.
C. Preparation of blend of granules obtained in step A and step B:
   1. Half quantity of Azilsartan medoxomil granules were mixed with Chlorthalidone granules;
   2. remaining granules of Azilsartan medoxomil were mixed in step 1 blend;
   3. microcrystalline cellulose and crospovidone were mixed in step 2;
   4. the blend obtained in step 3 was lubricated by using magnesium stearate;
   5. the lubricated blend was compressed into tablets in rotary compression machine; and
   6. the compressed tablets were coated with Opadry.

TABLE 15
Stability results of Example 05
Impurity Data

| | Azilsartan medoxomil | | | Chlorthalidone | | |
|---|---|---|---|---|---|---|
| | | 1 M | 6 M | | 1 M | 6 M |
| | | 40°/75% | 40°/75% | | 40°/75% | 40°/75% |
| Stage | Initial | RH | RH | Initial | RH | RH |

Azilsartan medoxomil Impurity

| | | | | |
|---|---|---|---|---|
| Total | 1.24 | 1.39 | 2.68 | NA |
| degradation | | | | |
| product for | | | | |
| Azilsartan | | | | |
| medoxomil | | | | |
| in % | | | | |

Chlorthalidone Impurity

| | | | | |
|---|---|---|---|---|
| Total | NA | 0 | 0 | 0 |
| degradation | | | | |
| product for | | | | |
| Chlorthalidone | | | | |
| in % | | | | |

### Example 6

TABLE 16
Composition comprising Azilsartan
kamedoxomil and Chlorthalidone:

| Sr. | | |
|---|---|---|
| No. | Ingredient | mg/tab |
| Intragranular Material | | |
| 1 | Azilsartan kamedoxomil | 42.680 |
| 2 | Mannitol | 162.640 |
| 3 | Microcrystalline cellulose | 36.000 |
| 4 | Crospovidone | 9.000 |
| Weight of Intragranules blend | | 250.32 |
| Binder | | |
| 5 | Fumaric acid | 4.000 |
| 6 | Sodium hydroxide | 2.000 |
| 7 | Hydroxy propyl cellulose | 6.000 |
| 8 | Purified water | Q.S. |
| Weight of dried granules | | 263.00 |
| Extragranular Material | | |
| 9 | Chlorthalidone | 25.000 |
| 10 | Microcrystalline cellulose | 30.000 |
| 11 | Crospovidone | 7.000 |
| 12 | Magnesium Stearate | 5.000 |
| Total weight of uncoated tablets | | 330.000 |
| 13 | Opadry | 10 |
| 14 | Purified Water | Q.S. |

| | |
|---|---|
| Total weight of coated tablets | 340.000 |

Manufacturing procedure of Example 06:
1. The intragranular materials (Azilsartan kamedoxomil, mannitol, microcrystalline cellulose and crospovidone) were granulated in fluidized bed granulator by using granulating solution, wherein the said granulating solution was prepared as follows:
   A. hydroxypropyl cellulose was added to purified water under continuous stirring. Stirring was continued till material was dissolved.
   B. fumaric acid and sodium hydroxide were added to purified water separately and then mixed and stirred to form clear solution;
2. the obtained granules were dried in fluidized bed processor to achieve desired LOD. The dried granules were passed through a 16 mesh sieve;
3. the extragranular material, microcrystalline cellulose (part quantity) was added to polybag containing Chlorthalidone and mixed;
4. the remaining part quantity of microcrystalline cellulose, half quantity dried granules and crospovidone were mixed with step 3 and further remaining quantity of dried granules were added;
5. the material obtained from step 4 was lubricated by using magnesium stearate;
6. the lubricated blend was compressed into tablets in rotary compression machine; and
7. the compressed tablets were coated with Opadry.

### Example 7

TABLE 17
Composition comprising Azilsartan
kamedoxomil and Chlorthalidone:

| Sr. | | |
|---|---|---|
| No. | Ingredient | mg/tab |
| Intragranular Material | | |
| 1 | Azilsartan kamedoxomil | 42.680 |
| 2 | Mannitol | 169.320 |
| 3 | Microcrystalline cellulose | 36.000 |
| 4 | Crospovidone | 9.000 |
| Weight of Intragranules blend | | 257.00 |
| Binder | | |
| 5 | Hydroxy propyl cellulose | 6.000 |
| 6 | Purified water | Q.S. |
| Weight of dried granules | | 263.00 |
| Extragranular Material | | |
| 7 | Chlorthalidone | 25.000 |
| 8 | Microcrystalline cellulose | 30.000 |
| 9 | Crospovidone | 7.000 |
| 10 | Magnesium Stearate | 5.000 |
| Total weight of uncoated tablets | | 330.000 |
| 11 | Opadry | 10 |
| 12 | Purified Water | Q.S. |

| | |
|---|---|
| Total weight of coated tablets | 340.000 |

Manufacturing procedure of Example 07:
01. The intragranular materials, Azilsartan kamedoxomil, mannitol, microcrystalline cellulose and crospovidone were added to FBP bowl and mixed for 5 minutes;
02. hydroxypropyl cellulose was added to purified water under continuous stirring and binder solution was prepared;
03. the binder solution obtained from step 02, was sprayed on material of step 01. Then binder solution of hydroxypropyl cellulose was sprayed on same material in fluidized bed processor;
04. the obtained granules were dried in fluidized bed processor to achieve desired LOD. The dried granules were passed through a 16 mesh sieve;
05. part quantity of microcrystalline cellulose was added to polybag containing Chlorthalidone and mixed;
06. the remaining part quantity of microcrystalline cellulose, half quantity dried granules and crospovidone was mixed with material of step 05;
07. the remaining quantity of dried granules were added to step 06 and mixed;
08. the blend obtained by step 07 was lubricated with magnesium stearate;
09. the lubricated blend was compressed into tablets in rotary compression machine; and
10. the compressed tablets were coated with Opadry.

### Example 8

TABLE 18
Composition comprising Azilsartan kamedoxomil:

| Sr. | | |
|---|---|---|
| No. | Ingredient | mg/tab |
| INTRAGRANULAR MATERIAL | | |
| 1 | Azilsartan kamedoxomil | 85.36 |
| 2 | Mannitol | 50-150 |
| 3 | Microcrystalline cellulose | 5-70 |
| 4 | Crospovidone | 4-20 |
| Weight of Intragranules blend | | About 293.34 |
| Binder | | |
| 5 | Fumaric acid | 10-16 |
| 6 | Sodium hydroxide | 2-12 |
| 7 | Hydroxy propyl cellulose/PVP K30 | 6-20 |
| 8 | Purified water | q.s. |
| EXTRAGRANULAR MATERIAL | | |
| 9 | Crospovidone | 4-20 |
| 10 | Magnesium Stearate | 3-6 |

| | |
|---|---|
| Total weight of tablet | 330.00 |

### Example 9

TABLE 19
Composition comprising Azilsartan kamedoxomil:

| Sr. | | |
|---|---|---|
| No. | Ingredient | mg/tab |
| INTRAGRANULAR MATERIAL | | |
| 1 | Azilsartan kamedoxomil | 85.36 |
| 2 | Mannitol | 121.98 |
| 3 | Microcrystalline cellulose | 66.00 |
| 4 | Crospovidone | 20.00 |
| Weight of Intragranular blend | | 293.34 |
| Binder | | |
| 5 | Fumaric acid | 12.00 |
| 6 | Sodium hydroxide | 8.04 |
| 7 | Hydroxy propyl cellulose | 8.00 |
| 8 | Purified water | |
| EXTRAGRANULAR MATERIAL | | |
| 9 | Crospovidone | 4.22 |
| 10 | Magnesium Stearate | 4.40 |

| | |
|---|---|
| Total weight of tablet | 330.00 |

### Example 10

TABLE 20
Composition comprising Azilsartan kamedoxomil:

| Sr. | | |
|---|---|---|
| No. | Ingredient | mg/tab |
| INTRAGRANULAR MATERIAL | | |
| 1 | Azilsartan kamedoxomil | 85.36 |
| 2 | Mannitol | 121.98 |
| 3 | Microcrystalline cellulose | 66.00 |
| 4 | Crospovidone | 20.00 |
| Weight of Intragranules blend | | 293.34 |
| Binder | | |
| 5 | Fumaric acid | 12.00 |
| 6 | Sodium hydroxide | 8.04 |
| 7 | PVP K30 | 8.00 |
| 8 | Purified water | |
| EXTRAGRANULAR MATERIAL | | |
| 9 | Crospovidone | 4.22 |
| 10 | Magnesium Stearate | 4.40 |

| | |
|---|---|
| Total weight of tablet | 330.00 |

### Manufacturing Procedure of Examples 08-10:

01. Dispensed all the excipient(s) accurately;
02. co-sifted intragranular materials (API-01, mannitol, microcrystalline cellulose and crospovidone) through 25 mesh sieve. Further, all the intragranular material was added to fluidized bed processor bowl and mixed for 10 minutes;
03. prepared binder solution in two steps; firstly, dissolved sodium hydroxide in purified water followed by fumaric acid and stirred continuously till all material was dissolved, and secondly, hydroxypropyl cellulose or PVP K30 was added to purified water under continuous stirring till material was dissolved;
04. the solution obtained from first part of step 03 was sprayed on the material of step 2. Further, binder solution obtained from second part of step 03 was sprayed on the same material in fluidized bed processor;
05. dried the material in fluidized bed processor to achieve desired LOD;
06. sifted dried granules through 18 mesh sieve;
07. required quantity of extragranular material (crospovidone) was taken according to dried granules yield and passed through 30 mesh sieve;
08. mixed the dried granules obtained from step 05 and material obtained from step 07 for 10 minutes in conta blender;
09. sifted magnesium stearate through 40 mesh sieve;
10. lubricated the blend obtained from step 09 with magnesium stearate for 3 minutes in conta blender;
11. compressed the blend obtained from step 10 in rotary compression machine and obtained tablets; and
12. the tablets obtained in step 11 were coated with Opadry coating solution.

TABLE 21
Stability Results of Example 10
Mono product - Batch similar to combo clinical, 3X FA and NaOH pH 5.5

| Stage | Initial | 1 M 40° C./75% RH |
|---|---|---|
| Total degradation product for Azilsartan | 0.93 | 1.38 |
| medoxomil in % | | |

### Example 11

TABLE 22
Composition comprising Azilsartan
kamedoxomil and Chlorthalidone:

| Sr. | | mg/tab for |
|---|---|---|
| No. | Ingredient | 40/25 mg |
| Azilsartan medoxomil Part | | |
| Intragranular Material | | |
| 1 | Azilsartan kamedoxomil | 42.680 |
| 2 | Mannitol | 62.900 |
| 3 | Micro crystalline cellulose | 33.000 |
| Weight of Intragranules blend | | 138.580 |
| Binder | | |
| 4 | Fumaric acid | 8.000 |
| 5 | Sodium hydroxide | 5.360 |
| 6 | HPC SSL | 4.000 |
| 7 | Purified Water | Q.S. |
| | Weight of Azilsartan medoxomil Granules | 155.940 |
| Chlorthalidone Part | | |
| Intragranular Material | | |
| 8 | Chlorthalidone | 25.000 |
| 9 | Mannitol | 98.960 |
| 10 | Crospovidone | 12.000 |
| 11 | Microcrystalline cellulose | 9.000 |
| Binder | | |
| 12 | PVP K 30 | 6.040 |
| 13 | Purified water | Q.S |
| | Weight of Chlorthalidone Granules | 151.000 |
| Extragranular Material | | |
| 14 | Azilsartan granules | 155.940 |
| 15 | Chlorthalidone granules | 151.000 |
| 16 | Crospovidone | 18.120 |
| 17 | Magnesium Stearate | 4.950 |
| Total weight of uncoated tablet | | 330.010 |
| Coating Part | | |
| 18 | Opadry Red | 9.900 |
| 19 | Purified water | Q.S. |

| | |
|---|---|
| Total weight of Coated tablet | 339.910 |

### Manufacturing Procedure of Examples 11:

A. Process for preparation of Chlorthalidone Granulation (part 1):
   1. Dispensed all the excipient(s) accurately;
   2. co-sifted intragranular material (Chlorthalidone, microcrystalline cellulose, part quantity of mannitol and crospovidone) through 18 mesh sieve and mixed into fluidized bed processor (FBP) bowl;
   3. dissolved PVP K 30 into purified water under continuous stirring;
   4. granulated the material of step 2 with step 3 material followed by drying of the granules, and
   5. passed the dried granules through 18 mesh sieve.
B. Process for preparation of Azilsartan medoxomil Granulation (part 2):
   6. Dispensed all the excipient(s) accurately;
   7. co-sifted of intragranular material (Azilsartan medoxomil, 80% of mannitol) through
   16 mesh sieve; and sifted microcrystalline cellulose through 18 mesh sieve;
   8. sifted remaining quantity of mannitol through 16 mesh sieve and added to fluidized bed processor bowl and mixed for 5 minutes;
   9. mixed the material of step no 2 and step no 3 for 5 minutes in FBP bowl;
   10. binder solution was prepared in two steps; firstly, sodium hydroxide was dissolved in purified water followed by fumaric acid and stirred till all material was dissolved, and secondly, added hydroxypropyl cellulose to purified water under continuous stirring till the material was dissolved;
   11. sprayed the solution obtained from first part of step 10 on the material in fluidized bed processor and then sprayed binder solution obtained from second part of step 10 on same material in fluidized bed processor;
   12. dried the material in fluidized bed processor to achieve desired LOD; and
   13. sifted the dried granules through 18 mesh sieve.
C. Blending with both API granules:
   14. Mixed Azilsartan medoxomil granules (part 1) with Chlorthalidone granules (part 2) for 20 minutes in conta blender;
   15. sifted crospovidone through 30 mesh sieve and mixed with material obtained from step 14 for 5 minutes;
   16. sifted magnesium stearate through 40 mesh sieve;
   17. lubricated the blend with magnesium stearate for 5 minutes in conta blender;
   18. compressed the blend obtained from step 17 into tablets in rotary compression machine; and
   19. prepared coating solution of Opadry in purified water and coated the compressed tablets.

Based on the above data, it was observed that the compositions of present invention exhibited excellent stability at accelerated and long term stability conditions.

1-10. (canceled)

11. A stable solid pharmaceutical composition comprising Azilsartan Kamedoxomil, optionally a diuretic, pH adjusting agent(s), and one or more pharmaceutically acceptable excipient(s) thereof.

12. The composition according to claim 11, wherein one or more pharmaceutically acceptable excipient(s) are selected from the group consisting of fillers, diluents, binders, disintegrants, coating agents, glidants, surfactants, lubricants, vehicle, and polymer or coating system.

13. The composition according to claim 11, wherein the pH adjusting agent comprises fumaric acid and sodium hydroxide base wherein the ratio of fumaric acid to sodium hydroxide base is about 1:0.2 to about 1:2.

14. The composition according to claim 11, wherein the diuretic is Chlorthalidone.

15. The composition according to claim 13, wherein the composition contains pH adjusting agent in 1% w/v solution of acid and base combination resulting in pH range from 5.1 to 7.0.

16. The composition according to claim 11, wherein the pH adjusting agent is present in the range from about 0.1% w/w to about 3% w/w of the composition.

17. A process for preparing solid pharmaceutical composition comprising Azilsartan or its salt and one or more pharmaceutically acceptable excipient(s) thereof; wherein the process comprises the steps of:
a) co-sifting intragranular material (API-01, diluent and disintegrant) through a sieve, adding material to fluidized bed processor bowl and mixing for 5 minutes;
b) dissolving binder and pH modifier in purified water under continuous stirring to constitute binder solution;
c) granulating material of step a) with the binder solution of step b) in fluidized bed processor followed by drying;
d) drying granules being passed through a sieve;
e) mixing dried granules and sifted extragranular material (diluent, disintegrant and surfactant) for 10 minutes in a conta blender;
f) passing lubricant through a sieve and lubricating the blend of step e) for 03 minutes in conta blender;
g) compressing lubricated blend of step f) into tablets in rotary compression machine; and
h) optionally coating the tablets of step g) with a solution comprising one or more coating agent and vehicle.

18. A process for preparing stable pharmaceutical composition comprising Azilsartan or its salt and one or more pharmaceutically acceptable excipient(s) thereof in combination with Chlorthalidone; wherein the process comprises steps of:
a) dispensing of all the excipient(s) accurately;
b) sifting of intragranular material (API-01 and API-02, mannitol, microcrystalline cellulose and crospovidone) and co-sifting of these excipient(s) through a sieve and adding of all the intragranular material to fluidized bed processor bowl and mixing for 10 minutes;
c) preparing binder solution in two steps; firstly dissolving sodium hydroxide in purified water followed by fumaric acid and stirring continuously till all material gets dissolved and secondly adding hydroxypropyl cellulose or PVP K30 to purified water under continuous stirring till material is dissolved;
d) spraying solution obtained from step (c) first part on material of step (b) and further, spraying of binder solution obtained from step (c) second part on the same material in fluidized bed processor;
e) drying of the material in fluidized bed processor to achieve desired LOD;
f) sifting of dried granules through a sieve;
g) weighing the extragranular material according to yield of dried granule and passing through a sieve;
h) mixing the dried granules obtained from step (f) and material obtained from step (g) for 10 minutes in conta blender;
i) sifting of magnesium stearate through a sieve;
j) lubricating the blend obtained from step (h) with magnesium stearate for 3 minutes in conta blender;
k) compressing the blend obtained from step (j) in rotary compression machine and obtaining tablets; and
l) optionally, coating the tablets of step (k) with a coating agent.
